(19) United States
(12) Patent Application Publication
(10) Pub. No.: US 2020/0337952 A1
(43) Pub. Date: Oct. 29, 2020

(54) OIL-BASED GEL COSMETIC

(71) Applicant: TOKIWA CORPORATION, Gifu (JP)

(72) Inventor: Shunsuke TOGASHI, Saitama (JP)

(73) Assignee: TOKIWA CORPORATION, Gifu (JP)

(21) Appl. No.: 16/848,141

(22) Filed: Apr. 14, 2020

(30) Foreign Application Priority Data
Apr. 25, 2019 (JP) 2019-083895

(51) Int. Cl. A61K 8/04; A61K 8/37; A61K 8/895; A61K 8/891; A61K 8/92

(52) U.S. Cl. CPC A61K 8/042; A61K 8/37; A61K 8/895; A61Q 1/04; A61K 8/92; A61K 2800/95; A61K 8/891

(57) ABSTRACT

To provide an oil-based gel cosmetic demonstrating well-spreading touch of use, and being balanced between high levels of long lastingness of cosmetic and shelf stability.

An oil-based gel cosmetic contains W a dextrin fatty acid ester having a degree of acylation of 2.0 or larger, (B) a crosslinked silicone elastomer having an alkyl group with 8 to 30 carbon atoms, and (C) a volatile oil.

### CROSS AREFERENCE TO RELATED APPLICATIONS

This application claims the benefit of Japanese Priority Patent Application JP-A-2019-083695 filed on Apr. 25, 2019, the entire contents of which are incorporated herein by reference.

### TECHNICAL FIELD

The present disclosure relates to an oil-based gel. cosmetic.

### RELATED ART

Oil-based cosmetics, having oil base, oil gelling agent and colorant blended therein, find their wide applications in makeup cosmetics including lip color, eyeliner and concealer. The oil gelling agent is blended for the purpose of retaining gelated state of the oil-based cosmetics or thickening the base oil, known materials for which are exemplified by wax, dextrin fatty acid ester, sucrose fatty acid ester, inulin fatty acid ester, metal soap such as aluminum 12-hydroxystearate, organic modified clay mineral such as Bentone, and partially crosslinked organopolysiloxane. Since poor compatibility between the oil gelling agent and the base oil results in separation of the base oil, or increases or decreases hardness of the cosmetics, so that it is an usual practice to choose an oil gelling agent most suitable for the base oil to be blended.

Investigations have been made on the base oil, aiming at enhancing the adhesiveness and improving the long lastingness of cosmetic, through addition of a volatile oil. JP-A-2014-080389 (Patent Document 1, below) discloses an exemplary oil-based cosmetic in which a volatile hydrocarbon base oil is gelated with a specific oil gelling agent.

### Citation List

Patent Document 1: JP-A-2014-080389

The cosmetics preferably have a proper gel hardness, from the viewpoint of obtaining a good touch of use represented by smooth spreadability when applied. The oil-based cosmetic described in JP-A-2014-080389, however, tends to change the gel hardness largely with time, showing insufficient stability of hardness. Another method has been proposed to stabilize the gel hardness by using a separate oil gelling agent such as wax or inulin fatty acid ester. The cosmetic, however, cannot retain therein the volatile oil when the gelated state collapses, upon being stirred from the need of filling the cosmetic into bottles or tubes, which unfortunately results in oil separation, difficulty of obtaining smooth spreadability, and degraded touch of use. Hence, the gel cosmetics containing the volatile oil are in need of further investigations to improve the shelf stability, while keeping the touch of use and usability at satisfactory levels.

The present disclosure was arrived at considering the aforementioned situations, an object of which is to provide an oil-based gel cosmetic demonstrating well-spreading touch of use, and being balanced between high levels of long lastingness of cosmetic and shelf stability.

### SUMMARY OF THE INVENTION

After our thorough investigations aimed at solving the aforementioned problem, the present inventors found that an oil-based gel cosmetic, prepared by blending two specific kinds of gelling agent and a volatile oil, successfully attained good spreadability, excellent long lastingness of cosmetic, and satisfactory levels of stability of gel hardness when observed with time and observed after stirring, and arrived at the present disclosure.

According to the present disclosure, there is provided an oil-based gel cosmetic that includes (A) a dextrin fatty acid ester having a degree of acylation of 2.0 or larger; (B) a crosslinked silicone elastomer having an alkyl group with 8 to 30 carbon atoms; and (C) a volatile oil.

With such structure, the oil-based gel cosmetic of the present disclosure is successfully balanced among well-spreading touch of use, long lastingness of cosmetic and shelf stability, all at high levels.

From the viewpoint of further improving transfer resistance, the oil-based gel cosmetic of the present disclosure preferably contains additionally (D) a silicone-based film forming agent.

From the viewpoint of balancing the spreadability and the gel stability at further higher levels, the oil-based gel cosmetic of the present disclosure preferably has a content of the dextrin fatty acid ester of 3 to 15% by mass, relative to the whole amount of the oil-based gel cosmetic.

The oil-based gel cosmetic of the present disclosure may contain dextrin palmitate as the dextrin fatty acid ester.

The oil-based gel cosmetic of the present disclosure may contain a hydrocarbon oil as the volatile oil.

### Advantageous Effects of the Invention

According to the present disclosure, it now becomes possible to provide an oil-based gel cosmetic that is successfully balanced among well-spreading touch of use, long lastingness of cosmetic and shelf stability, all at high levels.

### DESCRIPTION OF THE PREFERRED EMBODIMENT

An oil-based gel cosmetic of this embodiment contains (A) a dextrin in fatty acid ester having a degree of acylation of 2.0 or larger (may occasionally be referred to as (A) ingredient, hereinafter), (B) a crosslinked silicone elastomer having an alkyl group with 8 to 30 carbon atoms (may occasionally be referred to as (B) ingredient, hereinafter), and (C) a volatile oil (may occasionally be referred to as (C) ingredient, hereinafter).

In this patent specification, the degree of acylation of sugar fatty acid ester is given by an average number of moles of fatty acid that esterifies three hydroxy groups on a monosaccharide unit.

In this patent specification, the gelated state is defined by a hardness of 0.01 to 1.0 N, when measured using a rheometer (from Rheotech) with a 3-mm-diameter disk tip of the attachment, at a penetration speed of 6 cm/min, a penetration depth of 10 mm, at 25° C.

### (A) Ingredient

The dextrin fatty acid ester haying a degree of acylation of 2.0 or larger is exemplified by those having a structure represented by formula (1) below:

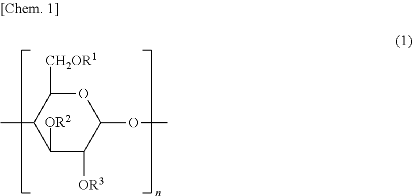

[in formula (1), each of R¹, R² and ^{R3} independently represents a hydrogen atom or R—CO— (R represents a straight-chain or branched, saturated or unsaturated hydrocarbon group), and n represents the degree of polymerization].

The upper limit of the degree of acylation is not specifically limited, but it may be 3.0 or less, further 2.5 or less.

The aforementioned R preferably has 8 to 24 carbon atoms, the number is more preferably 14 to 18. The aforementioned n preferably represents 3 to 150, which is more preferably 10 to 100, and even more preferably 15 to 40. The dextrin fatty acid ester having a degree of acylation of 2.0 or larger may have a structure in which at least two of R¹, R² and R³ in formula (1) represent R—CO—.

The dextrin fatty acid ester is exemplified by dextrin palmitate, dextrin 2-ethylhexanoate, dextrin stearate, dextrin oleate, dextrin isonalmitate, dextrin isostarate, and dextrin (palmitate/ethylhexanoate).

The (A) ingredient is available under the product names Rheopearl KL2, Rheopearl KS2 and Rheopearl MKL2 (all from Chiba Flour Milling Co., Ltd.).

Only a single kind of the (A) ingredient may be used, or two or more kinds may be used in a combined manner.

The oil-based gel cosmetic of this embodiment may have a content of the (A) ingredient, relative to the whole amount of the oil-based gel cosmetic, of 2% by mass or more, which is preferably 3% by mass or more. Also, the content of the (A) ingredient, relative to the whole amount of the oil-based gel cosmetic, is preferably 15% by mass or less, more preferably 13% by mass or less, further more preferably 11% by mass or less.

### (B) Ingredient

The crosslinked silicone elastomer having an alkyl group with 8 to 30 carbon atoms is exemplified by a compound composed of a crosslinked three-dimensional, silicone-based elastomer having an alkyl group with 8 to 30 carbon atoms. The alkyl group with 8 to 30 carbon atoms preferably has a straight-chain structure, and is exemplified by lauryl group, myristoyl group, palmitoyl group, and stearoyl group. The crosslinked silicone elastomer preferably contains vinyl group.

The (B) ingredient is typically composed of at least one structural unit selected from the group consisting of SiO₂ unit, HSiO_{1.5} unit, RSiO_{2.5} unit, RHSiO unit, R₂SiO unit, R₃SiO_{0.5} unit and R₂HSiO_{0.5} unit, and is obtainable by making an organohydrogen polysiloxane, having 1.5 hydrogen atoms on average bound to each silicon atom, addition-polymerized with a vinyl group-containing organopolysilcxane having a structural unit selected from the group consisting of SiO₂ unit, (CH₂═CH)SiO_{1.5} unit, RSiO_{1.5} unit, R(CH₂═CH)SiO unit, R₂SiO unit, R₃SiO_{0.5} unit and R₂(CH₂═CH)SiO_{0.5} unit, and having in the molecule 1.5 vinyl groups on average bound to each silicon atom in the molecule, and/or, with an unsaturated hydrocarbon represented by CₘH₂ₘ₋₁(CH₂)ₓCₘH₂ₘ₋₁ (where, m represents an integer of 2 to 6, and x represents an integer of 1 or larger). Each R represents a substituted or unsubstituted monovalent hydrocarbon group having 1 to 30 carbon atoms, but excluding aliphatic unsaturated group, and a part of such R represents an alkyl group with 8 to 30 carbon atoms. The alkyl group with 8 to 30 carbon atoms may accounts for 5 to 50 mol % of the structural units, which may even be 10 to 40 mol %.

The crosslinked silicone elastomer is exemplified by (vinyl dimethicone/lauryl dimethicone) crosspolymer, (lauryl polydimethylsiloxyethyl dimethicone/bisvinyl dimethicone) crosspolymer, (PEG-15/lauryl dimethicone) crosspolymer, (PEG-10/lauryl dimethicone) crosspolymer, (PEG-15/lauryl dimethicone) crosspolymer, (PEG-15/lauryl polydimethylsiloxyethyl dimethicone) crosspolymer, (lauryl dimethicone/polyglycerin-3) crosspolymer, and (polyglyceryl-3/lauryl polydimethylsiloxyethyl dimethicone) crosspolymer.

Among the crosslinked silicone elastomer, (vinyl dimethicone/lauryl dimethicone) crosspolymer is preferable.

The crosslinked silicone elastomer in the oil-based gel cosmetic of this embodiment may be blended in the form of mixture dispersed in a dispersion medium,

The dispersion medium is exemplified by hydrocarbon oil, ester oil, and silicone oil.

The hydrocarbon oil is exemplified by the hydrocarbon oil previously exemplified as the (C) ingredient, mineral oil, squalane, and olefin oligomer. The ester oil is exemplified by triethylhexanoin, isotridecyl isononanoate, and neopentyl glycol diethylhexanoate. The silicone oil is exemplified by dimethicone, diphenyisiloxyphenyl trimethicone, cyclopentasiloxane, and methyl trimethicone.

The mixture may have any swelled form such as gel or paste. The mixture is also commercially available. Examples of the commercial products are as follows.

Examples include KSG-41A (mixture of (vinyl dimethicone/lauryl dimethicone) crosspolymer and mineral oil, elastomer ingredient: ca. 25% by mass); KSG-42A. (mixture of (vinyl dimethicone/lauryl dimethicone) crosspolymer and isododecane, elastomer ingredient: ca. 20% by mass); KSG-42 (mixture of (vinyl dimethicone/lauryl dimethicone) crosspolymer and isododecane, elastomer ingredient: ca. 25% by mass); KSG-43 (mixture of (vinyl dimethicone/lauryl dimethicone) crosspolymer and triethylhexanoin, elastomer ingredient: ca. 30% by mass); KSG-44 (mixture of (vinyl dimethicone/lauryl dimethicone) crosspolymer and squalane, elastomer ingredient: ca. 30% by mass); KSG-0422 (mixture of (lauryl polydimethylsiloxyethyl dimethicone/bisvinyl dimethicone) crosspolymer and isododecane, elastomer ingredient: ca. 20% by mass); KSG-045Z, (mixture of (lauryl polydimethylsiloxyethyl dimethicone/bisvinyl dimethicone) crosspolymer and cyclopentasiloxane, elastomer ingredient: ca. 20% by mass); KSG-048Z (mixture of (lauryl polydimethylsiloxyethyl dimethicone/bisvinyl dimethicone) crosspolymer and dimethicone, elastomer ingredient: ca. 20% by mass); KSG-310 (mixture of (PEG-15/lauryl dimethicone) crosspolymer and mineral oil, elastomer ingredient: ca. 30% by mass); KSG-320 (mixture of (PFG-15/lauryl dimethicone) crosspolymer and isododecane, elastomer ingredient: ca. 25% by mass); KSG-330 (mixture of (PEG-15/lauryl dimethicone) crosspolymer and. triethylhexanoin, elastomer ingredient: ca. 17.5% by mass); KSG-340 (mixture of (PEG-10/lauryl dimethicone) crosspolymer and (PEG-15/lauryl dimethicone) crosspolymer and squalane, elastomer ingredient: ca. 30% by mass); KSG-320Z (mixture of (PEG-15/lauryl polydimethylsiloxyethyl dimethicone) crosspolymer and isododecane, elastomer ingredient: ca. 25% by mass); KSG-350Z (mixture of (PEG-15/lauryl polydimethylsiloxyethyl dimethicone) crosspolymer and cyclopentasiloxane, elastomer ingredient: ca. 25% by mass); KSG-360Z (mixture of (PEG-15/lauryl polydimethylsiloxyethyl dimethicone) crosspolymer and dimethicone, elastomer ingredient ca. 35% by mass); KSG-380Z (mixture of (PEG-15/lauryl polydimethylsiloxyethyl dimethicone) crosspolymer and dimethicone, elastomer ingredient: ca. 30% by mass); KSG-810 (mixture of (lauryl dimethicone/polyglycerin-3) crosspolymer and mineral oil, elastomer ingredient: ca. 30% by mass); KSG-820 (mixture of (lauryl dimethicone/polyglycerin-3) crosspolymer and isododecane, elastomer ingredient: ca. 25% by mass); KSG-830 (mixture of (lauryl dimethicone/polyglycerin-3) crosspolymer and triethylhexanoin, elastomer ingredient: ca. 17.5% by mass); KSG-840 (mixture of (lauryl dimethicone/polyglycerin-3) crosspolymer and squalane, elastomer ingredient: ca. 30% by mass); KSG-820Z (mixture of (polyglyceryl-3/lauryl polydimethylsiloxyethyl dimethicone) crosspolymer and isododecane, elastomer ingredient: ca. 25% by mass); and, KSG-850Z, (mixture of (polyglyceryl-3/lauryl polydimethylsiloxyethyl dimethicone) crosspolymer and cyclopentasiloxane, elastomer ingredient: ca. 25% by mass) (all from Shin-Etsu Chemical Co., Ltd.).

The content of the (B) ingredient (elastomer ingredient) in the oil-based gel cosmetic of this embodiment may be 3 to 15% by mass, relative to the whole amount of the oil-based gel cosmetic, which is preferably 3 to 10% by mass from the viewpoints of usability and gel stability, and is more preferably 4 to 8% by mass.

From the viewpoint of gel stability, the total content of the (A) ingredient and the (B) ingredient preferably accounts for 6 to 30% by mass of the whole amount of the oil-based gel cosmetic, which is more preferably 10 to 25% by mass.

The oil-based gel cosmetic of this embodiment is easily adjustable in terms of gel hardness by varying the amounts of blending of the (A) ingredient and the (B) ingredient, can easily improve not only the touch of use but also matching with containers and application tools, can easily be filled in containers, and can easily be taken up by an applicator.

### (C) Ingredient

The volatile oil employable here is hydrocarbon oil or silicone oil. The volatile oil means a base oil having a boiling point of 250° C. or lower at 1 atm (101.325 kPa).

The volatile hydrocarbon oil is exemplified by isododecane, hydrogenated polyisobutene, coconut alkanes, and isohexadecane.

The volatile silicone oil is exemplified by methyl trimethicone, dimethicone (1cs), dimethicone (1.5cs), dimethicone (2cs), cyclopentasiloxane, cyclohexasiloxane, and caprylyl methicone.

The volatile oil preferably has a boiling point at 1 atom of 230° C. or lower, which is more preferably 210° C. or lower, even more preferably 205° C. or lower, and yet more preferably 200° C. or lower. Although the lower limit is not specifically limited, the lower limit may be 50° C. or more.

The content of the (C) ingredient in the oil-based gel cosmetic of this embodiment may be 10 to 80% by mass, relative to the whole amount of the oil-based gel cosmetic, which is more preferably 15 to 70% by mass from the viewpoints of long lastingness of cosmetic and gel stability, and even more preferably 20 to 60% by mass.

The mass ratio of the total amount of the (B) crosslinked silicone elastomer and the (C) volatile oil to the (A) dextrin. fatty acid ester ((A)/[(B)+(C)]) is preferably 0.02 or more, more preferably 0.03 or more, further more preferably 0.05 or more, particularly preferably 0.1 or more. And the upper limit of (A)/[(B)+(C)] is preferably 1.15 or less, more preferably 1.0 or less, further more preferably 0.85 or less, particularly preferably 0.5 or less. By setting the above preferable range, the effect of the invention is more remarkably exerted.

The oil-based gel cosmetic of this embodiment may further contain the (D) silicone-based film forming agent (occasionally referred to as (D) ingredient, hereinafter), from the viewpoint of improving the transfer resistance.

The silicone-based film forming agent is exemplified by trimethylsiloxysilicate, acryl-modified silicone, polypropylsilsesquioxane, polymethylsilsesquioxane, polyphenyisilsesquioxane, and (acrylates/methacrylic acid polytrimethylsiloxy) copolymer.

The content of the (D) ingredient in the oil-based gel cosmetic of this embodiment may be 3 to 30% by mass relative to the whole amount of the oil-based gel cosmetic, which is preferably 3 to 20% by mass from the viewpoint of gel stability, and even more preferably 3 to 15% by mass.

The oil-based gel cosmetic of this embodiment may further contain a powdery ingredient, a gelling agent other than the (A) ingredient and (B) ingredient, a base oil other than the (C) ingredient, or a surfactant.

The powdery ingredient may be any of those usually used for cosmetics without special limitation, and is exemplified by extender powder, glitter powder, white pigment, and coloring pigment. The powder may also have unspecified morphology, and may have a spherical, plate-like or needle-like shape; a grain size of fume-grade, particle-grade or pigment-grade; or a porous or non-porous particle structure.

Specific examples include extender powders such as talc, mica, synthetic mica, sericite, kaolin, silicon carbide, barium sulfate, bentonite, smectite, aluminum oxide, silica, magnesium oxide, zirconium oxide, magnesium carbonate, calcium carbonate, chromium oxide, and aluminum magnesium hydroxide; glitter powders such as bismuth oxychloride, mica titanium, titanium dioxide coated bismuth oxychloride, iron oxide coated mica, iron oxide coated mica titanium, red iron oxide coated mica titanium, red iron oxide/black iron oxide coated mica titanium, organic pigment coated mica titanium, argentine, iron oxide coated synthetic phlogopite, aluminum flake, titanium dioxide coated glass flake, polyethylene terephthalate/aluminum/epoxy laminated film powder, and polyethylene terephthalate/polyolefin laminated film powder; ultraviolet scattering agents such as titanium oxide fine particle and zinc oxide fine particle; organic powders such as nylon powder, polymethyl methacrylate powder, acrylonitrile-methacrylic acid copolymer powder, vinylidene chloride-methacrylic acid copolymer, polyethylene powder, polystyrene powder, organopolysiloxane elastomer powder, polymethylsilsesquioxane powder, urethane powder, wool powder, silk powder, cellulose powder and N-acyllysine powder; composite powders such as pulverized titanium oxide coated mica titanium, pulverized titanium oxide coated nylon, barium sulfate coated mica titanium, titanium oxide-containing silica and zinc oxide-containing silica; and metal soaps such as magnesium stearate, zinc myristate, aluminum stearate and calcium stearate.

The coloring pigment is exemplified by inorganic coloring pigments such as red iron oxide, yellow iron oxide, black iron oxide, cobalt oxide, chromium oxide, ultramarine, Prussian blue, titanium oxide and zinc oxide; organic coloring pigments such as Red No. 228, Red No. 226, Blue No. 404, Red No. 201, Red No. 202 and Yellow No. 4-aluminum lake; and natural colors such as carmine and safflower.

Only one kind of the powdery ingredient may be used independently, or two or more kinds are used in a combined manner.

The content of the powdery ingredient may be 1 to 60% by mass, relative to the whole amount of the oil-based gel cosmetic, which is preferably 2 to 50% by mass from the viewpoint of touch of use.

The gelling agent other than the (A) ingredient and (B) ingredient employable here includes sugar fatty acid esters such as dextrin fatty acid ester having a degree of acylation of smaller than 2.0 (for example, dextrin palmitate, etc.), and stearoyl inulin; crosslinked silicone elastomer free of alkyl group with 8 to 30 carbon atoms; metal soaps such as aluminum stearate, magnesium stearate, zinc stearate, aluminum myristate, magnesium myristate, zinc myristate, zinc palmitate, magnesium laurate and zinc laurate; organic modified clay mineral such as disteardimonium hectorite; amino acid-based gelling agents such as dibutyl ethylhexanoyl giutamide and dibutyllauroyl glutamide; fine particle silica; and 12-hydroxystearic acid.

The base oil other than the (C) ingredient employable here may be any of those usually used for cosmetics, without special limitation, which may be solid oil, and base oil other than solid oil. Only one kind of the base oil other than the (C) ingredient may be used independently, or two or more kinds are used in a combined manner.

The solid oil is exemplified by hydrocarbons such as paraffin wax, microcrystalline wax, and polyethylene; plant-derived oils and fats such as hydrogenated castor oil, hydrogenated jojoba oil, carnauba wax, and rice bran wax; esters such as glyceryl trilbehenate and cholesterol fatty acid ester; higher fatty acids such as stearic acid and behenic acid; higher alcohols such as stearyl alcohol and behenyl alcohol; and silicones such as alkyl-modified silicone and acryl-modified silicone. Only one kind of these compounds may be used independently, or two or more kinds are used in a combined manner.

The base oil other than the solid oil employable here may be pasty oil and liquid oil. The pasty oil is exemplified by vaseline, dipentaerythrityl hexa(hydroxystearate/stearate/rosinate), dipentaerythrityl tetra(hydroxystearate/isostearate), dipentaerythrityl pentaisostearate, dipentaerythrityl hexahydroxystearate, caprylic/capric/myristic/stearic triglyceride, hydrogenated castor oil isostearate, phytosteryl oleate, sucrose hexa(oleate/palmitate/stearate), bis(behenyl/isostearyl/phytosteryl) dimer dilinoleyl dimer dilinoleate, (phytosteryl/isostearyl/cetyl/stearyl/behenyl) dimer dilinoleate, hydrogenated castor oil dimer dilinoleate, phytosteryl macadamiate, and bis-diglyceryl polyaeyladipate-2.

The liquid oil is exemplified by ester oils such as cetyl ethylhexanoate, ethylhexyl palmitate, triethylhexanoin, isotridecyl isononanoate, isostearyl isostearate, neopentyl glycol diethylhexanoate, glyceryl tri(caprylate/caprate), neopentyl glycol dicaprate, propanediol isostearate, trimethylolpropane triethylhexanoate, octyldcdecyl myristate, octyldodecyl stearoyloxystearate, diisostearyl malate, polyglyceryl triisostearate, dipentaerythrithyl pentaisostearate, and trimethylolpropane triisostearate; silicone oils such as dimethicone and methylphenylpolysiloxane; hydrocarbon oils such as liquid paraffin, squalane, and olefin oligomer; vegetable oils such as sunflower seed oil, jojoba seed oil, olive oil, and castor oil; higher fatty acids such as isostearic acid; and higher alcohols such as isostearyl alcohol, octyl dodecanol, and oleyl alcohol.

The oil-based gel cosmetic of this embodiment may contain a surfactant, from the viewpoint of workability when dispersing the powdery ingredient.

The surfactant employable here may be hydrophilic nonionic surfactant, anionic surfactant, cationic surfactant, or amphoteric surfactant. The hydrophilic nonionic surfactant is exemplified by polyoxyalkylene alkyl ether, glycerin alkyl ether, glycerin fatty acid ester, polyglycerin fatty acid ester, sorbitan fatty acid ester, and alkylene glycol adducts of them, polyalkylene glycol fatty acid ester, polyglycerin modified silicone, and polyether modified silicone. The anionic surfactant is exemplified by alkylphosphate, polyoxyalkylene alkyl ether phosphate, sulfonate, alkylsulfate, and polyaspartate. The cationic surfactant is exemplified by alkylamine salt and alkyltrimethylammonium salt. The amphoteric surfactant is exemplified by lecithin, carbobetaine-type amphoteric surfactant, sulfobetaine-type amphoteric surfactant, and amino acid-type amphoteric surfactant. Only one kind of the surfactant may be used independently, or two or more kinds are used in a combined manner.

The oil-based gel cosmetic of this embodiment may contain, besides the aforementioned ingredients, any of ingredients usually used for cosmetics, such as preservative, antioxidant, colorant, thickener, pH adjuster, fragrance, UV absorber, UV scattering agent, moisturizer, chelating agent, and anti-inflammatory.

The oil-based gel cosmetic of this embodiment preferably has a hardness of 1.0 N or smaller when measured using a rheometer (from Rheotech) with a 3-mm-diameter disk tip of the attachment, at a penetration speed of 6 cm/min, a penetration depth of 10 mm, at 25° C. from the viewpoint of touch of use, which is preferably 0.6 N or smaller, meanwhile it is preferably 0.01 N or larger from the viewpoint of gel stability, and is more preferably 0.03 N or larger.

The oil-based gel cosmetic of this embodiment is suitable for makeup cosmetics such as concealer, foundation, eyeliner, cheek color, and lip color.

Next, a method for manufacturing the oil-based gel cosmetic of this embodiment will be explained.

The method for manufacturing the oil-based gel cosmetic of this embodiment includes preparing a cosmetic base that contains the aforementioned (A) ingredient, (B) ingredient, (C) ingredient, and optionally contains the (D) ingredient, powdery ingredient and other ingredient; and filling the cosmetic base in a predetermined container and gelating it.

The cosmetic base may have a chemical composition suitably adjusted so that the aforementioned oil-based gel cosmetic of this embodiment will be obtainable. Also the ratio of blending of the individual ingredients in the cosmetic base may fall in the preferred ranges preset for the aforementioned oil-based gel cosmetic.

The method for preparing the cosmetic base may be exemplified by a method mixing the aforementioned (A) ingredient, (D) ingredient, (C) ingredient, and the optional (D) ingredient, powdery ingredient, and other ingredient.

The mixing may be implemented typically by mixing the oil-based ingredients ((A) ingredient, (B) ingredient, (C) ingredient, and optional (D) ingredient, and other base oil) under heating to obtain a mixture, and then by mixing the thus obtained mixture further with the powdery ingredient and the other ingredient.

Mixing may be achieved by using a three-roll mill, disper mixer, or homomixer.

Gelation of the cosmetic base may be achieved typically by cooling the filled cosmetic base down to room temperature.

The predetermined container is exemplified by stick container, bottle, tube, jar, and pouch container.

### Example

The present disclosure will now be further detailed referring to Examples. Note, however, that. Examples by no means limit the technical scope of the present disclosure. All numerals given in Tables represent contents (% by mass) relative to the whole amount of the cosmetic base.

Before describing Examples, evaluation methods employed in the individual Examples will be explained.

### (1) Touch of Use (Spreadability) and Long Lastingness of Cosmetic Minesiveness)

Twenty expert panelists on cosmetic evaluation were individually asked to use sample cosmetics, and to rate the spreadability and adhesiveness of each cosmetic according to a five-grade scale below. Scores from all panelists were averaged and then rated according to the criteria below.

### Evaluation Criteria

5: Very good

### 4: Good

### 3: Fair

### 2: Poor

1: Very poor

### Evaluation Criteria for Spreadability (Averaged Score)

⊚: 4≤
∘: 3≤ and <4
Δ: 2≤ and <3
x: <2

### Evaluation Criteria for Adhesiveness (Averaged Score)

⊚: 4≤
∘: 3≤ and <4
Δ: 2≤ and <3
x: <2

### (2) Transfer Resistance

Each cosmetic was uniformly spread over an 1 cm×3 cm area of BIOSKIN plate (from. Beaulax Co., Ltd.) to form an applied area. Ten minutes after, paper was pressed on the applied area, and adhesion of the cosmetic was visually evaluated according to the four-grade below.

### Evaluation Criteria

⊚: No adhesion on paper
∘: Slight adhesion on paper
Δ: ⅓ to ½ of spread cosmetic adhered on paper
x: ½ or more of spread cosmetic adhered on paper

### (3) Temporal Stability of Hardness

Initial hardness was measured using a rheometer (from Rheotech) with a 3-mm-diameter disk tip of the attachment, at a penetration. speed of 6 cm/min, a penetration. depth of 10 mm, at 25° C. The cosmetic was then stored at 25° C. for one month, and the hardness after storage was measured in the same way. Rate of change of hardness, relative to the initial hardness, was determined, and was rated according to the two-grade criteria below.

### Evaluation Criteria

∘: Rate of change of hardness<50%
x: Rate of change of hardness≥50%

### (4) Gel Stability

Each cosmetic in the container was stirred, stored at 40° C. for one month, change of appearance was observed, and rated according to the three-grade criteria below.

### Evaluation Criteria

⊚: No change
∘: Slight oil separation observed
x: Distinct oil separation as much causing outflow from inclined container

### Examples 1 to 12 and Comparative Examples 1 to 7: Eyeliner

Eyeliners having chemical compositions summarized in Tables 1 to 3 were prepared according to the method described below, filled in stick containers, and evaluated in the same way as described above. Also results are collectively summarized in Tables 1 to 3.

### Manufacturing Method

The ingredients other than the powdery ingredient were melted and mixed under heating up to 100° C. The powdery ingredient was then added to the mixture, and uniformly dispersed to obtain a cosmetic base. The obtained cosmetic base was filled in the stick container, and cooled down to room temperature to obtain each gel eyeliner.

TABLE 1

| | | Example | | | |
|---|---|---|---|---|---|
| | | 1 | 2 | 3 | 4 |
| (A) Ingredient | Dextrin palmitate A | 8 | 10 | 8 | 8 |
| Fixed oil | Neopentyl glycol | 8 | 8 | 8 | 8 |
| | diethylhexanoate | | | | |
| (C) Ingredient | Isododecane | 18.4 | 19.4 | — | — |
| | Hydrogenated | — | — | 18.4 | — |
| | polyisobutene | | | | |
| | Coconut alkanes | — | — | — | 18.4 |
| (D) Ingredient | Trimethylsiloxysilicate | 10 | 10 | 10 | 10 |
| (B) Ingredient + | Silicone elastomer A | 25 | 25 | 25 | 25 |
| (C) Ingredient | | | | | |
| Fixed oil | Defoaming agent | 0.05 | 0.05 | 0.05 | 0.05 |
| | Antioxidant | 0.05 | 0.05 | 0.05 | 0.05 |
| | Preservative | 0.5 | 0.5 | 0.5 | 0.5 |
| Powder | Synthetic mica | 5 | — | 5 | 5 |
| | Black iron oxide | 25 | 27 | 25 | 25 |
| Evaluation | Use (spreadability) | ⊚ | ⊚ | ⊚ | ⊚ |
| | Long lastingness of | ⊚ | ⊚ | ⊚ | ◯ |
| | cosmetic (adhesiveness) | | | | |
| | Transfer resistance | ⊚ | ⊚ | ⊚ | ⊚ |
| | Temporal stability | ◯ | ◯ | ◯ | ◯ |
| | of hardness | | | | |
| | Gel stability | ⊚ | ⊚ | ⊚ | ⊚ |
| Hardness of the cosmetics (N) | | 0.24 | 0.42 | 0.22 | 0.33 |
| | | Example | | | |
| | | 5 | 6 | 7 | 8 |
| (A) Ingredient | Dextrin palmitate A | 3 | 15 | 10 | 8 |
| Fixed oil | Neopentyl glycol | 8 | 8 | 8 | — |
| | diethylhexanoate | | | | |
| (C) Ingredient | Isododecane | 23.4 | 11.4 | 27.4 | 1.4 |
| | Hydrogenated | — | — | — | — |
| | polyisobutene | | | | |
| | Coconut alkanes | — | — | — | — |
| (D) Ingredient | Trimethylsiloxysilicate | 10 | 10 | 10 | 10 |
| (B) Ingredient + | Silicone elastomer A | 25 | 25 | 25 | 25 |
| (C) Ingredient | | | | | |
| Fixed oil | Defoaming agent | 0.05 | 0.05 | 0.05 | 0.05 |
| | Antioxidant | 0.05 | 0.05 | 0.05 | 0.05 |
| | Preservative | 0.5 | 0.5 | 0.5 | 0.5 |
| Powder | Synthetic mica | 5 | 5 | 5 | 5 |
| | Black iron oxide | 25 | 25 | 27 | 15 |
| Evaluation | Use (spreadability) | ⊚ | ◯ | ⊚ | ◯ |
| | Long lastingness of | ⊚ | ⊚ | ⊚ | ⊚ |
| | cosmetic (adhesiveness) | | | | |
| | Transfer resistance | ⊚ | ⊚ | ⊚ | ⊚ |
| | Temporal stability | ◯ | ◯ | ◯ | ◯ |
| | of hardness | | | | |
| | Gel stability | ◯ | ⊚ | ◯ | ⊚ |

| | | | | |
|---|---|---|---|---|
| Hardness of the cosmetic (N) | 0.10 | 0.78 | 0.18 | 0.68 |

TABLE 2

| | | Example | | | |
|---|---|---|---|---|---|
| | | 9 | 10 | 11 | 12 |
| (A) Ingredient | Dextrin palmitate A | 3 | 15 | 8 | 8 |
| Fixed oil | Neopentyl glycol | 8 | — | 16.4 | — |
| | diethylhexanoate | | | | |
| (C) Ingredient | Isododecane | 36.4 | 4.4 | 10 | 61.4 |
| (D) Ingredient | Trimethylsiloxysilicate | 10 | — | 10 | — |
| (B) Ingredient + | Silicone elastomer A | 12 | 60 | — | 25 |
| (C) Ingredient | | | | | |
| (B) Ingredient + | Silicone elastomer B | — | — | 25 | — |
| fixed oil | | | | | |
| Fixed oil | Defoaming agent | 0.05 | 0.05 | 0.05 | 0.05 |
| | Antioxident | 0.05 | 0.05 | 0.05 | 0.05 |
| | Preservative | 0.5 | 0.5 | 0.5 | 0.5 |
| Powder | Synthetic mica | 5 | — | 5 | — |
| | Black iron oxide | 25 | 20 | 25 | 5 |
| Evaluation | Use (spreadability) | ⊚ | ◯ | ⊚ | ⊚ |
| | Long lastingness of | ⊚ | ◯ | ◯ | ◯ |
| | cosmetic (adhesiveness) | | | | |
| | Transfer resistance | ⊚ | ◯ | ⊚ | ◯ |
| | Temporal stability | ◯ | ◯ | ◯ | ◯ |
| | of hardness | | | | |
| | Gel stability | ◯ | ⊚ | ⊚ | ⊚ |

| | | | | |
|---|---|---|---|---|
| Hardness of the cosmetics (N) | 0.05 | 0.98 | 0.51 | 0.08 |

TABLE 3

| | | Comparative example | | | |
|---|---|---|---|---|---|
| | | 1 | 2 | 3 | 4 |
| (A) Ingredient | Dextrin palmitate A | 8 | 8 | — | — |
| Gelling agent | Dextrin palmitate B | — | — | — | 10 |
| | Stearoyl inulin | — | — | — | — |
| | Polyethelene wax | — | 5 | 5 | — |
| Fixed oil | Neopentyl glycol | 8 | — | 8 | 8 |
| | diethylhexanoate | | | | |
| | Diisostearyl malate | — | — | — | — |
| | Dimethicone | — | — | — | — |
| (C) Ingredient | Isododecane | 43.4 | 44.4 | 28 | 19.4 |
| (D) Ingredient | Trimethylsiloxysilicate | 10 | 12 | 12 | 10 |
| (B) Ingredient + | Silicone elastomer A | — | — | 25 | 25 |
| (C) Ingredient | | | | | |
| (B) Ingredient + | Silicone elastomer B | — | — | — | — |
| fixed oil | | | | | |
| (C) Ingredient + | Silicone elastomer C | — | — | — | — |
| elastomer | | | | | |
| Fixed oil | Defoaming agent | 0.05 | 0.05 | 0.05 | 0.05 |
| | Antioxidant | 0.05 | 0.05 | 0.05 | 0.05 |
| | Preservative | 0.5 | 0.5 | 0.5 | 0.5 |
| Powder | Synthetic mica | 5 | — | 1.4 | — |
| | Black iron oxide | 25 | 30 | 20 | 27 |
| Evaluation | Use (spreadability) | ⊚ | Δ | X | ⊚ |
| | Long lastingness of | Δ | Δ | ⊚ | ⊚ |
| | cosmetic (adhesiveness) | | | | |
| | Transfer resistance | Δ | Δ | ⊚ | ⊚ |
| | Temporal stability | ◯ | ◯ | ◯ | X |
| | of hardness | | | | |
| | Gel stability | X | X | X | ◯ |

| | | | | |
|---|---|---|---|---|
| Hardness of the cosmetics (N) | 0.05 | 0.1 | unmeasurable | 0.16 |

| | | Comparative example | | |
|---|---|---|---|---|
| | | 5 | 6 | 7 |
| (A) Ingredient | Dextrin palmitate A | — | 8 | 8 |
| Gelling agent | Dextrin palmitate B | — | — | — |
| | Stearoyl inulin | 10 | — | — |
| | Polyethylene wax | — | — | — |
| Fixed oil | Neopentyl glycol | 8 | 16.4 | — |
| | diethylhexanoate | | | |
| | Diisostearyl malate | — | 1 | — |
| | Dimethicone | — | 8 | — |
| (C) Ingredient | Isododecane | 19.4 | — | 14.4 |
| (D) Ingredient | Trimethylsiloxysilicate | 10 | 12 | 12 |
| (B) Ingredient + | Silicone elastomer A | 25 | — | — |
| (C) Ingredient | | | | |
| (B) Ingredient + | Silicone elastomer B | — | 24 | — |
| fixed oil | | | | |
| (C) Ingredient + | Silicone elastomer C | — | — | 35 |
| elastomer | | | | |
| Fixed oil | Defoaming agent | 0.05 | 0.05 | 0.05 |
| | Antioxidant | 0.05 | 0.05 | 0.05 |
| | Preservative | 0.5 | 0.5 | 0.5 |
| Powder | Synthetic mica | — | — | — |
| | Black iron oxide | 27 | 30 | 30 |
| Evaluation | Use (spreadability) | ◯ | ⊚ | ⊚ |
| | Long lastingness of | ⊚ | X | ⊚ |
| | cosmetic (adhesiveness) | | | |
| | Transfer resistance | ⊚ | X | ⊚ |
| | Temporal stability | ◯ | ◯ | ◯ |
| | of hardness | | | |
| | Gel stability | X | ◯ | X |

| | | | |
|---|---|---|---|
| Hardness of the cosmetics (N) | unmeasurable | 0.55 | unmeasurable |

Details of the individual ingredients summarized in Tables 1 to 3, and in Table 4 shown later, will be listed below.

Dextrin palmitate A: Rheopearl KL2 (degree of acylation=2.2) (from Chiba Flour Killing Co., Ltd., product name)
Dextrin palmitate 5: Rheopearl TL2 (degree of acylation=1.6) (from Chiba. Flour Milling Co., Ltd., product name)
Stearoyl inulin: Rheopearl ISK2 (degree of acylation≥2.2) (from Chiba Flour Milling Co., Ltd., product name)
trimethylsiloxysilicate: BELSIL TMS 803 (from Wacker Asahikasei Silicone Co., Ltd., product name)
Silicone elastomer A: mixture of 25% by mass of KSG-42 (vinyl dimethicone/lauryl dimethicone) crosspolymer and 75% by mass of isododecane (from Shin-Etsu Chemical Co., Ltd., product name)
Silicone elastomer B: mixture of 30% by mass of KSG-43 (vinyl dimethicone/lauryl dimethicone) crosspolymer and 70% by mass of triethylhexanoin (from Shin-Etsu Chemical. Co., Ltd., product name)
Silicone elastomer C: mixture of 17.5% by mass of KSG-1610 (dimethicone/vinyl dimethicone) crosspolymer and 82.5% by mass of methyl trimethicone (from Shin-Etsu Chemical Co., Ltd., product name)
Silicone elastomer D: mixture of 25% by mass of KSG-320 (PEG-15/lauryl dimethicone) crosspolymer and 75% by mass of isododecane (from Shin-Etsu Chemical Co., Ltd., product name)

As summarized in Tables 1 and 2, the oil-based gel cosmetics obtained in Examples 1 to 12 were confirmed to be worthy of good evaluation regarding all of touch of use (spreadability), long lastingness of cosmetic (adhesiveness), transfer resistance, temporal stability of hardness, and gel stability.

### Examples 13 to 17: Concealer and Lip Color

A concealer (Example 13) and lip colors (Example 14 to 17) having chemical compositions listed in Table 4 were prepared. The concealer and the lip colors in Examples 14 to 16 were filled in bottles. The lip color in Example 17 was filled in a tube. Also results of evaluation are collectively summarized in Table 4.

### Manufacturing Method

The ingredients other than the powdery ingredient were melted and mixed under heating up to 100° C. The powdery ingredient was then added to the mixture, and uniformly dispersed to obtain a cosmetic base. The obtained cosmetic base was filled in the container, and cooled down to room temperature to obtain the gel concealer or each gel lip color.

TABLE 4

| | | Example | | |
|---|---|---|---|---|
| | | 13 | 14 | 15 |
| (A) Ingredient | Dextrin palmitate A | 5.5 | 7 | 7 |
| Gelling agent | Dextrin palmitate B | 3 | 3 | 3 |
| Fixed oil | Isotridecyl isononanoate | 5 | — | — |
| | Diisostearyl malate | 1 | 7.55 | 12.55 |
| | Diphenylsiloxyphenyl trimethicone | — | 30 | 25 |
| (C) Ingredient | Isododecane | 18.9 | 10 | 15 |
| | Methyl trimethicone | — | — | — |
| (D) Ingredient | Trimethylsiloxysilicate | 12 | — | — |
| (B) Ingredient + | Silicone elastomer A | — | 30 | 25 |
| (C) Ingredient | Silicone elastomer D | 20 | — | — |
| Fixed oil | Defoaming agent | 0.05 | 0.05 | 0.05 |
| | Antioxidant | 0.05 | 0.05 | 0.05 |
| | Preservative | 0.5 | 0.5 | 0.5 |
| Powder | Synthetic mica | 1 | 3 | 3 |
| | Titanium oxide | 25 | 1 | 1 |
| | Yellow iron oxide | 6 | — | — |
| | Red iron oxide | 1 | — | — |
| | Black iron oxide | 1 | — | — |
| | Mica titanium | — | 7 | 7 |
| | Red 202 | — | 0.3 | 0.3 |
| | Yellow 4 | — | 1 | 1 |
| Evaluation | Use (spreadability) | ⊚ | ⊚ | ⊚ |
| | Long lastingness of | ⊚ | ⊚ | ⊚ |
| | cosmetic (adhesiveness) | | | |
| | Transfer resistance | ⊚ | ◯ | ◯ |
| | Temporal stability | ◯ | ◯ | ◯ |
| | of hardness | | | |
| | Gel stability | ⊚ | ⊚ | ⊚ |

| | | | |
|---|---|---|---|
| Hardness of the cosmetics (N) | 0.14 | 0.32 | 0.28 |

| | | Example | |
|---|---|---|---|
| | | 16 | 17 |
| (A) Ingredient | Dextrin palmitate A | 7 | 6 |
| Gelling agent | Dextrin palmitate B | 3 | 3 |
| Fixed oil | Isotridecyl isononanoate | — | — |
| | Diisostearyl malate | — | 12.55 |
| | Diphenylsiloxyphenyl trimethicone | — | 26 |
| (C) Ingredient | Isododecane | 57.55 | — |
| | Methyl trimethicone | — | 10 |
| (D) Ingredient | Trimethylsiloxysilicate | — | — |
| (B) Ingredient + | Silicone elastomer A | 20 | 30 |
| (C) Ingredient | Silicone elastomer D | — | — |
| Fixed oil | Defoaming agent | 0.05 | 0.05 |
| | Antioxidant | 0.05 | 0.05 |
| | Preservative | 0.5 | 0.5 |
| Powder | Synthetic mica | 3 | 3 |
| | Titanium oxide | 1 | 1 |
| | Yellow iron oxide | — | — |
| | Red iron oxide | — | — |
| | Black iron oxide | — | — |
| | Mica titanium | 7 | 7 |
| | Red 202 | 0.3 | 0.3 |
| | Yellow 4 | 1 | 1 |
| Evaluation | Use (spreadability) | ⊚ | ⊚ |
| | Long lastingness of | ⊚ | ⊚ |
| | cosmetic (adhesiveness) | | |
| | Transfer resistance | ◯ | ◯ |
| | Temporal stability | ◯ | ◯ |
| | of hardness | | |
| | Gel stability | ⊚ | ⊚ |

| | | |
|---|---|---|
| Hardness of the cosmetics (N) | 0.12 | 0.33 |

As summarized in Table 4, the oil-based gel cosmetics obtained in Examples 13 to 17 were confirmed to be worthy of good evaluation regarding all of touch of use (spreadability), long lastingness of cosmetic (adhesiveness), temporal stability of hardness, and gel stability. The oil-based gel cosmetic obtained in Example 13 was found to be worthy of good evaluation also regarding transfer resistance.

### Example 18: Mascara

| (Ingredient) | (Blending ratio (% by mass)) |
|---|---|
| 1. Dextrin palmitate A | 6.00 |
| 2. Neopentyl glycol diethylhexanoate | 3.50 |
| 3. Diisostearyl malate | 1.00 |
| 4. Methyl trimethicone | 22.30 |
| 5. Trimethylsiloxysilicate | 9.60 |
| 6. Silicone elastomer B | 25.00 |
| 7. Defoaming agent | 0.05 |
| 8. Antioxidant | 0.05 |
| 9. Preservative | 0.50 |
| 10. Titanium oxide | 1.00 |
| 11. Black iron oxide | 1.80 |
| 12. Yellow iron oxide | 0.50 |
| 13. Red iron oxide | 0.70 |
| 14. Synthetic mica | 20.00 |
| 15. PMMA | 8.00 |

Details of the individual ingredients are same as described previously.

### Manufacturing Method

The ingredients other than the powdery ingredient were melted and mixed under heating up to 100° C. The powdery ingredient was then added to the mixture, and uniformly dispersed to obtain a cosmetic base. The obtained cosmetic base was filled in a container, and cooled down to room temperature to obtain a mascara. The mascara was found to have a hardness at 25° C. of 0.40 N.

### Evaluation

The thus obtained mascara, evaluated in the same way as described above, was found to show touch of use (spreadability) represented by “⊚”, long lastingness of cosmetic (adhesiveness) represented by “⊚”, transfer resistance represented by “⊚”, temporal stability of hardness represented by “∘”, and gel stability represented by “⊚”.

### Example 19: Mascara

| (Ingredient) | (Blending ratio (% by mass) |
|---|---|
| 1. Dextrin palmitate A | 2.00 |
| 2. Dextrin palmitate B | 3.00 |
| 3. Neopentyl glycol diethylhexanoate | 5.50 |
| 4. Diisostearyl malate | 1.20 |
| 5. Methyl trimethicone | 22.30 |
| 6. Trimethylsiloxysilicate | 9.60 |
| 7. Silicone elastomer B | 25.00 |
| 8. Defoaming agent | 0.05 |
| 9. Antioxidant | 0.05 |
| 10. Preservative | 0.50 |
| 11. Titanium oxide | 1.00 |
| 12. Black iron oxide | 1.60 |
| 13. Yellow iron oxide | 0.50 |
| 14. Red iron oxide | 0.70 |
| 15. Mica | 12.00 |
| 16. PMMA | 15.00 |

Details of the individual ingredients are same as described previously.

### Manufacturing Method

The ingredients other than the powdery ingredient were melted and mixed under heating up to 100° C. The powdery ingredient was then added to the mixture, and uniformly dispersed to obtain a cosmetic base. The obtained cosmetic base was filled in a container, and cooled down to room temperature to obtain. a mascara. The mascara was found to have a hardness at 25° C. of 0.15 N.

### Evaluation

The thus obtained mascara, evaluated in the same way as described. above, was found to show touch of use (spreadability) represented by “⊚”, long lastingness of cosmetic (adhesiveness) represented by “⊚”, transfer resistance represented by “⊚”, temporal stability of hardness represented by “∘”, and gel stability represented by “∘”.

It should be understood. by those skilled in the art that various modifications, combinations, sub-combinations and alterations may occur depending on design requirements and other factors insofar as they are within the scope of the appended claims or the equivalents thereof.

What is claimed is:

1. An oil-based gel. cosmetic comprising:
a dextrin fatty acid ester having a degree of acylation of 2.0 or larger;
(B) a crosslinked silicone elastomer having an alkyl group with 8 to 30 carbon. atoms; and
(C) a volatile oil.

2. The oil-based gel. cosmetic of claim 1, further comprising (D) a silicone-based film forming agent.

3. The oil-based gel cosmetic of claim 1, wherein content of the dextrin fatty acid ester is 2 to 15% by mass, relative to the whole amount of the oil-based gel cosmetic.

4. The oil-based gel cosmetic of claim 1, wherein the dextrin fatty acid ester is dextrin palmitate.

5. The oil-based gel cosmetic of claim 1, wherein the volatile oil is a hydrocarbon oil.

6. The oil-based gel cosmetic of claim 1, wherein content of the crosslinked. silicone elastomer is 3 to 15% by mass, relative to the whole amount of the oil-based gel cosmetic.

7. The oil-based gel cosmetic of claim 1, wherein the dextrin fatty acid ester has a structure represented by formula (1); 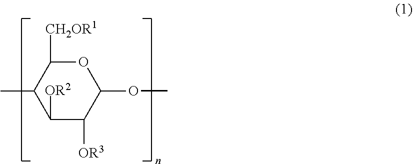
wherein each of R¹, R² and R³ independently represents a hydrogen atom or R—CO—, where R represents a straight-chain or branched, saturated or unsaturated hydrocarbon group; and n represents a degree of polymerization.

8. The oil-based gel cosmetic of claim 1, wherein the crosslinked silicone elastomer contains a vinyl group.

9. The oil-based gel cosmetic of claim 1, wherein the crosslinked silicone elastomer contains (vinyl dimethicone/lauryl dimethicone) crosspolymer.

10. The oil-based gel cosmetic of claim 2, wherein content of the dextrin fatty acid ester is 2 to 15% by mass, relative to the whole amount of the oil-based gel cosmetic.

11. The oil-based gel cosmetic of claim 2, wherein the dextrin fatty acid ester is dextrin palmitate.

12. The oil-based gel cosmetic of claim 2, wherein the volatile oil is a. hydrocarbon oil.

13. The oil-based gel cosmetic of claim 2, wherein content of the crosslinked. silicone elastomer is 3 to 15% by mass, relative to the whole amount of the oil-based gel cosmetic.

14. The oil-based gel cosmetic of claim 2, wherein the dextrin fatty acid ester has a structure represented by formula (1); 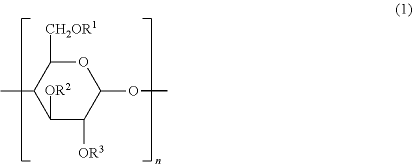
wherein each of R¹, R² and R³ independently represents a hydrogen atom or R—CO—, where R represents a straight-chain or branched, saturated or unsaturated hydrocarbon group; and n represents a degree of polymerization.

15. The oil-based gel cosmetic of claim 3, wherein the dextrin fatty acid ester is dextrin palmitate.

16. The of gel cosmetic of claim 3, wherein the volatile oil is a hydrocarbon oil.

17. The oil-based gel cosmetic of claim 3, wherein content of the crosslinked silicone elastomer is 3 to 15% by mass, relative to the whole amount of the oil-based gel cosmetic.

18. The oil-based gel cosmetic of claim 3, wherein the dextrin fatty acid ester has a structure represented by formula (1); 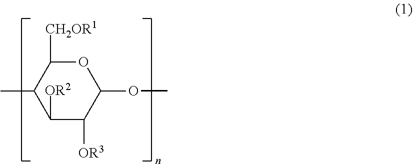
wherein each of R¹, R² and R³ independently represents a hydrogen atom or R—CO—, where R represents a straight-chain or branched, saturated or unsaturated hydrocarbon group; and n represents a degree of polymerization.

19. The oil-based gel cosmetic of claim 4, wherein the volatile oil is a hydrocarbon oil.

20. The oil-based gel cosmetic of claim 19, wherein the crosslinked silicone elastomer contains (vinyl dimethicone/lauryl dimethicone) crosspolymer.
